(19) United States
(12) Patent Application Publication
(10) Pub. No.: US 2021/0313054 A1
(43) Pub. Date: Oct. 7, 2021

(54) HOSPITAL SYSTEM, SERVER DEVICE, AND METHOD OF MANAGING SCHEDULE

(71) Applicant: Sony Corporation, Tokyo (JP)

(72) Inventors: Yasuaki TAKAHASHI, Tokyo (JP); Tsuneo HAYASHI, Tokyo (JP); Kenji TAKAHASHI, Tokyo (JP); Koji KASHIMA, Tokyo (JP); Keisuke UYAMA, Tokyo (JP)

(73) Assignee: Sony Corporation, Tokyo (JP)

(21) Appl. No.: 17/267,032

(22) PCT Filed: Sep. 6, 2019
(86) PCT No.: PCT/JP2019/035135

§ 371 Date: Feb. 9, 2021

(30) Foreign Application Priority Data
Sep. 11, 2018 (JP) 2018-169812

(51) Int. Cl. G16H 40/20; G16H 40/60; G06Q 10/06

(52) U.S. Cl. CPC G16H 40/20; G06Q 10/063114; G16H 40/60

(57) ABSTRACT

The present disclosure relates to a hospital system, a server device, and a method of managing a schedule that enable an operation schedule to be updated properly.

A schedule management unit manages the operation schedule, a presentation unit presents the operation schedule, and a status estimation unit estimates a status of an operation on the basis of a type of the operation currently in progress and resource information from a resource related to the operation. Moreover, the schedule management unit reflects the estimated status of the operation in the operation schedule, and the presentation unit presents the operation schedule in which the status of the operation is reflected. The present disclosure is applicable to, for example, a hospital system.

### TECHNICAL FIELD

The present disclosure relates to a hospital system, a server device, and a method of managing a schedule, and more particularly to a hospital system, a server device, and a method of managing a schedule that enable an operation schedule to be updated properly.

### BACKGROUND ART

In medical institutions, medical workers make operation schedules. At that time, the operation schedules need to be made efficiently in consideration of various factors such as a type of the operation, time required for the operation, a schedule of surgeons who can perform the operation, a schedule of operating rooms, and a schedule of medical instruments required for the operation.

For example, Patent Document 1 discloses a system that assigns radio-frequency identification (RFID) to medical instruments and monitors positional information and usage statuses of the medical instruments to efficiently operate and manage the medical instruments.

### CITATION LIST

### Patent Document

Patent Document 1: Japanese Patent Application Laid-Open No. 2013-97761

### SUMMARY OF THE INVENTION

### Problems to be Solved by the Invention

Meanwhile, in a case where a significant extension occurs in an operation, a created operation schedule is required to be updated properly.

The present disclosure has been conceived in view of such a situation, and it is intended to enable an operation schedule to be updated properly.

### Solutions to Problems

A hospital system according to the present disclosure includes a schedule management unit that manages an operation schedule, a presentation unit that presents the operation schedule, and a status estimation unit that estimates, on the basis of a type of an operation currently in progress and resource information from a resource related to the operation, a status of the operation, in which the schedule management unit reflects the estimated status of the operation in the operation schedule, and the presentation unit presents the operation schedule in which the status of the operation is reflected.

A server device according to the present disclosure includes a schedule management unit that manages an operation schedule, and a status estimation unit that estimates, on the basis of a type of an operation currently in progress and resource information from a resource related to the operation, a status of the operation, in which the schedule management unit reflects the estimated status of the operation in the operation schedule.

A method of managing a schedule according to the present disclosure causes a server device that manages an operation schedule to estimate, on the basis of a type of an operation currently in progress and resource information from a resource related to the operation, a status of the operation, and to reflect the estimated status of the operation in the operation schedule.

In the present disclosure, the operation status is estimated on the basis of a type of the operation currently in progress and resource information from a resource related to the operation, and the estimated operation status is reflected in the operation schedule.

### BRIEF DESCRIPTION OF DRAWINGS

FIG. 1 is a diagram illustrating an exemplary configuration of a hospital system according to the present embodiment.

FIG. 2 is a flowchart illustrating a flow of changing of an operation schedule.

FIG. 3 is a flowchart illustrating details of the changing of the operation schedule.

FIG. 4 is a diagram illustrating exemplary operation schedule presentation.

FIG. 5 is a diagram illustrating exemplary operation schedule presentation.

FIG. 6 is a diagram illustrating exemplary operation schedule presentation.

FIG. 7 is a diagram illustrating exemplary operation schedule presentation.

FIG. 8 is a diagram illustrating another exemplary configuration of the hospital system.

FIG. 9 is a diagram illustrating still another exemplary configuration of the hospital system.

FIG. 10 is a block diagram illustrating an exemplary hardware configuration of a server device.

Hereinafter, a mode for carrying out the present disclosure (hereinafter referred to as an embodiment) will be described. Note that descriptions will be given in the following order.

1. System Configuration

2. Flow of Operation Schedule Changing

3. Exemplary Presentation of Operation Schedule

4. Variations

5. Hardware Configuration

6. Summary

<1. System Configuration>

In a hospital system according to the present embodiment, an operation schedule in a hospital is updated in real time according to a progress in an operation, a status of resources (room, equipment, and manpower) related to the operation, and the like.

For example, the following information is sensed as a status of resources.

1. Room (operating room, laboratory, nearby medical facility, etc.)

(A) Operational status: in use, standby state, in preparation, cleanup, vacancy, etc.

(B) Condition: existence of defects, etc.

2. Equipment (device, surgical instrument, inspection equipment, wiring, etc.)

(A) Operational status: in use, standby state, sterilization, maintenance, vacancy, etc.

(B) Condition: existence of defects, remaining battery level, operating time, etc.

(C) Location: placement in room, hospital room, ambulance, nearby medical facility, etc.

3. Manpower (doctor, anesthesiologist, pathologist, medical worker such as nurse)

(A) Job status: working, on a break, being out, etc.

(B) Condition: working time, physical condition, etc.

(C) Location: inside hospital, outside hospital, etc.

In the hospital system according to the present embodiment, an operation schedule is updated on the basis of a status of sensed resources and a type of an operation currently in progress.

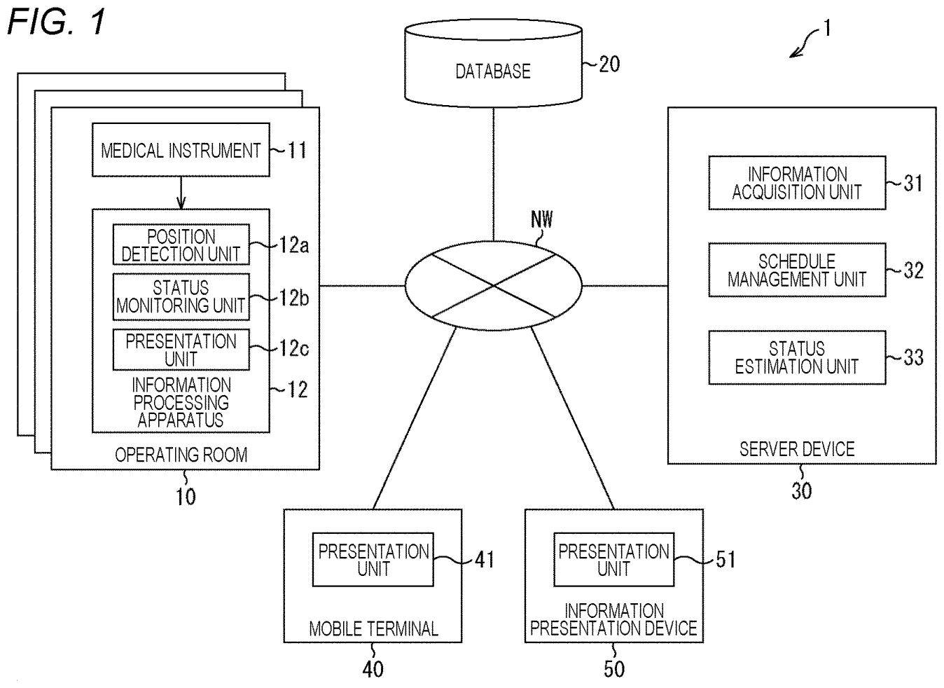

FIG. 1 is a diagram illustrating an exemplary configuration of the hospital system according to the present embodiment.

A hospital system 1 in FIG. 1 illustrates a configuration of one hospital as a whole. A status of resources (room, equipment, and manpower) related to an operation is managed in the hospital system 1.

The hospital system 1 includes an operating room 10, a database 20, a server device 30, a mobile terminal 40, and an information presentation device 50.

The operating room 10, the database 20, the server device 30, the mobile terminal 40, and the information presentation device 50 are each connected by a network NW. The network NW may be configured by the Internet, or may be configured by a local area network (LAN) in the hospital.

For example, the operating room 10, the database 20, and the server device 30 are connected to the network NW by wire, and the mobile terminal 40 and the information presentation device 50 are connected to the network NW wirelessly.

(Operating Room)

The operating room 10 is a room for operations, and is provided with an information processing apparatus 12 that senses a status of each resource including equipment such as a medical instrument 11.

The information processing apparatus 12 includes a position detection unit 12a, a status monitoring unit 12b, and a presentation unit 12c.

The position detection unit 12a detects a position of the medical instrument 11.

The status monitoring unit 12b monitors a status of the medical instrument 11.

For example, the status monitoring unit 12b monitors the status of the medical instrument 11 by analyzing video input from the medical instrument 11 and measuring a current consumption of the medical instrument 11.

Furthermore, the status monitoring unit 12b monitors the status of the medical instrument 11 on the basis of an image captured by a camera (not illustrated), or communicates with the medical instrument 11 according to a predetermined communication protocol to monitor the status of the medical instrument 11.

The information processing apparatus 12 transmits, to the server device 30, resource information related to the resources such as the position of the medical instrument 11 detected by the position detection unit 12a and the status of the medical instrument 11 monitored by the status monitoring unit 12b via the network NW.

The presentation unit 12c presents an operation schedule and various kinds of information related to the operation schedule to medical workers in the operating room 10, such as doctors and nurses, using images and sounds.

For example, the presentation unit 12c displays an operation schedule and the arrangement plan of equipment according to the operation schedule on the basis of the information transmitted from the server device 30 via the network NW, and displays a warning in a case where the equipment is not arranged in the correct operating room.

Furthermore, in a case where the operation schedule is changed, the presentation unit 12c may display a changed operation schedule, and in a case where the equipment is transferred due to the change of the operation schedule, it may display the destination of the equipment.

(Database)

The database 20 retains an operation schedule generated in advance, information associated with patients, work information of doctors, nurses, and the like, equipment management information, information associated with past operations, information associated with nearby medical facilities, and the like. Those pieces of information are updated or read out by the server device 30 as appropriate.

(Server Device)

The server device 30 manages the operation schedule retained in the database 20 on the basis of the status of each resource sensed by the information processing apparatus 12.

The operation schedule is associated with information indicating, for each operation performed in each room (operating room) in the hospital, each resource such as the scheduled time, the surgeon to perform the operation, and the medical instruments to be used for the operation.

The server device 30 includes an information acquisition unit 31, a schedule management unit 32, and a status estimation unit 33.

The information acquisition unit 31 obtains the resource information transmitted from the information processing apparatus 12, and also obtains, from the resource information, information indicating the type of the operation currently in progress. The type of the operation may be obtained by identifying the operation being performed at the current time from the operation schedule.

The schedule management unit 32 manages the operation schedule retained in the database 20.

The status estimation unit 33 estimates a status of the operation currently in progress on the basis of the type of the operation currently in progress obtained by the information acquisition unit 31 and the resource information transmitted from the information processing apparatus 12.

The schedule management unit 32 reflects the operation status estimated by the status estimation unit 33 in the operation schedule. For example, the schedule management unit 32 changes the operation schedule depending on the estimated operation status.

The operation schedule retained in the database 20 is updated to reflect the operation status.

The operation schedule managed by the schedule management unit 32 is transmitted to the mobile terminal 40, the information presentation device 50, and the presentation unit 12c of the information processing apparatus 12 described above.

(Mobile Terminal/Information Presentation Device)

The mobile terminal 40 is a tablet terminal or a mobile phone (smartphone) carried by a medical worker in the hospital.

The presentation unit 41 included in the mobile terminal 40 presents the operation schedule managed by the schedule management unit 32.

The information presentation device 50 is a monitor installed in the hospital. The presentation unit 51 included in the information presentation device 50 also presents the operation schedule managed by the schedule management unit 32.

The presentation unit 41 and the presentation unit 51 include a display, a speaker, and the like, and present an operation schedule, instructions to medical workers based on a change in the operation schedule, and the like by images and sounds. Furthermore, the presentation unit 12c of the information processing apparatus 12 also presents an operation schedule, instructions to medical workers based on a change in the operation schedule, and the like by images and sounds. Moreover, an operation schedule, instructions to medical workers based on a change in the operation schedule, and the like may be presented by machine voices via an in-house announcement or an extension, and a string of letters directed to the mobile terminal 40 and the information presentation device 50 via an e-mail or a message application.

Note that the instructions to medical workers based on a change in the operation schedule may be given only to the resources affected by the change in the operation schedule.

Furthermore, in a case where a type of the operation is input to the presentation unit 41, the presentation unit 51, and the presentation unit 12c, the resources required for the operation (medical instruments such as an endoscope and medical materials such as gauze) may be presented by images and voices. Moreover, in a case where positional information of each resource is obtained, the existing position of the resource required for the operation may be presented on the basis of the positional information of the resource.

<2. Flow of Operation Schedule Changing>

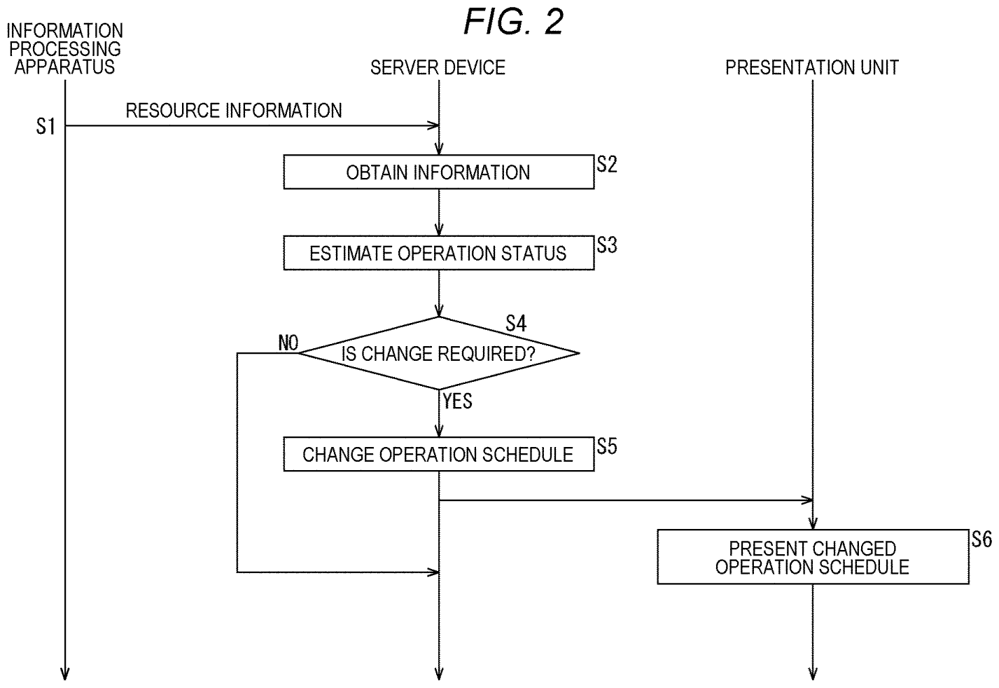

In the following descriptions, the overall flow of operation schedule changing in the hospital system 1 described above will be described with reference to FIG. 2. The process of FIG. 2 may be performed at regular time intervals, or may be triggered by a change in a status of resources sensed by the information processing apparatus 12 or generation of a request from the server device 30.

In step S1, the information processing apparatus 12 transmits resource information such as a position and a status of the medical instrument 11 to the server device 30.

In step S2, the information acquisition unit 31 of the server device 30 obtains the resource information from the information processing apparatus 12, and also obtains a type of the operation from the resource information.

Next, in step S3, the status estimation unit 33 of the server device 30 estimates a status of the operation currently in progress on the basis of the obtained operation type and the resource information.

In step S4, the schedule management unit 32 of the server device 30 determines whether or not the operation schedule needs to be changed from the estimated operation status.

If it is determined that the operation schedule needs to be changed, in step S5, the schedule management unit 32 of the server device 30 changes the operation schedule according to the estimated operation status.

In step S6, the presentation unit 41 of the mobile terminal 40, the presentation unit 51 of the information presentation device 50, and the presentation unit 12c of the information processing apparatus 12 present the changed operation schedule.

The operation schedule is changed according to the flow as described above.

Meanwhile, if it is determined in step S4 that the operation schedule does not need to be changed, the schedule management unit 32 of the server device 30 does not change the operation schedule, and is standby until the information processing apparatus 12 transmits the resource information next time.

(Details of Operation Schedule Changing)

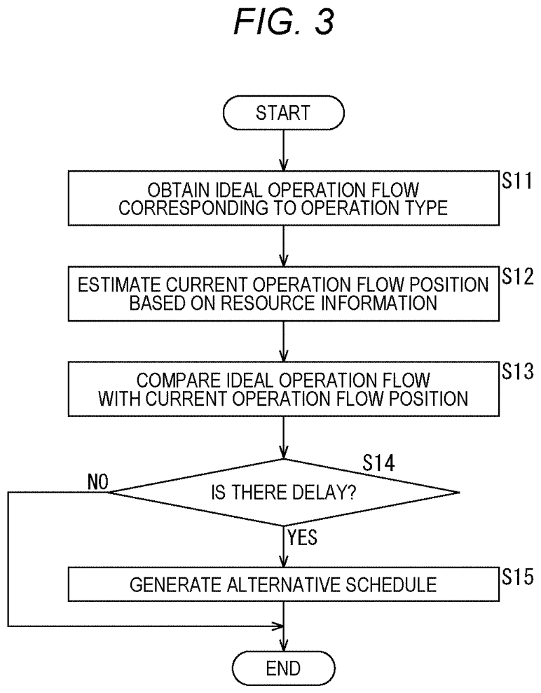

Next, details of the operation schedule changing performed by the schedule management unit 32 of the server device 30 will be described with reference to the flowchart of FIG. 3.

In step S11, the schedule management unit 32 obtains an ideal operation flow corresponding to the type of the operation currently in progress from the database 20.

A procedure of a general operation is determined by an operative method, and the ideal operation flow is information indicating the procedure of the operation and the standard time required for each procedure. The ideal operation flow for each operation type is registered in the database 20. The schedule management unit 32 obtains the ideal operation flow of the items corresponding to the name of the operation currently in progress by searching an “operation name/operation flow table” in the database 20.

In step S12, the schedule management unit 32 estimates the current operation flow position on the basis of the resource information. The operation flow position is a current temporal position in the operation flow of the operation currently in progress.

Equipment information from the medical instrument 11 to be used for the operation is used as the resource information.

For example, the schedule management unit 32 inputs the image captured by an operative field camera, which is obtained as equipment information, into a machine learning model learned from the learning data of the operation flow, thereby estimating a current operation flow position.

The schedule management unit 32 may use ON/OFF information of medical instruments as the equipment information. In this case, a current operation flow position is estimated from the medical instrument being used (in the ON state).

Furthermore, input information input by a medical worker such as a doctor and a nurse may be used as resource information.

For example, the schedule management unit 32 may use a voice instruction from a medical worker as input information. In this case, the voice instruction from the operator is subject to voice recognition, and a current operation flow position is estimated on the basis of the obtained instruction content.

In step S13, the schedule management unit 32 compares the ideal operation flow with the estimated current operation flow position.

In step S14, the schedule management unit 32 determines whether or not the current operation flow position is delayed compared to the standard time indicated by the ideal operation flow. At this time, for example, the current operation flow position may be compared with the standard time with information specific to the doctor in charge of the operation being taken into consideration.

In a case where the current operation flow position is determined to be behind the ideal operation flow, in step S15, the current operation schedule is changed to generate an alternative schedule. As a result, an operation schedule that reflects the operation flow position is to be presented.

Meanwhile, in a case where the current operation flow position is determined not to be behind the ideal operation flow in step S14, no alternative schedule is generated (current operation schedule is not changed), and the process is terminated.

According to the process described above, it becomes possible to update the operation schedule appropriately even in a case where a significant extension occurs in the operation.

(Generation of Alternative Schedule)

Here, a specific example of the alternative schedule generation mentioned above will be described.

In a case where a certain operation is delayed in the operation schedule, the schedule management unit 32 delays the start of the operation to be performed next (next operation) in the operating room where the operation is being performed, or changes the operation schedule such that the next operation will be performed in another operating room that is vacant. Furthermore, the schedule management unit 32 also changes the operation schedule in a case where the equipment and person involved in the delayed operation will be involved in the operation to be performed in the other operating room.

The operation schedule is changed on the basis of a priority level of the next operation itself and the resources (room, equipment, and manpower) to be involved in the operation.

It is assumed that the priority level is higher as a value of the score representing the priority level becomes smaller, for example. In a case where the score is zero, the operation can be started. The score can also take a negative value, and in that case, a priority level becomes higher as an absolute value is higher.

Scores for a room, equipment, and manpower are set by quantifying the time required to start the operation with the room, equipment, and manpower, or by quantifying the shortest time required to make the lacking equipment and manpower available. That is, a priority level is determined according to a status of resources related to the next operation.

Furthermore, the priority level of the operation itself may be input by a medical worker in advance as a score. The score in this case is set to be a negative value, and may be changed as appropriate on the day of the operation.

In the operation schedule, operations with scores set in this manner are reflected in the schedule in ascending order of the score. Note that only the start time is changed with regard to an operation that cannot be performed in another operating room due to the facilities in the operating room or the like. However, the operation that cannot be performed in another operating room may be transferred to another operating room by manual input of a medical worker.

In the process of FIG. 3, it is determined whether or not the current operation flow position is delayed by comparing the current operation flow position with the ideal operation flow while the operation is being performed.

In addition to this, for example, it may be determined whether or not the current operation flow position is delayed depending on whether or not the medical worker has input the operation end by the scheduled operation end time indicated by the ideal operation flow.

For example, in a case where the medical worker does not input the operation end by the scheduled operation end time, the current operation flow position may be determined to be delayed, and the operation schedule may be changed. Furthermore, the operation schedule may be changed also in a case where the medical worker has input the operation end before the scheduled operation end time.

While the changed operation schedule (alternative schedule) is presented to each medical worker, it may be presented only to a manager who manages the operation progress, among the medical workers.

In that case, the changed operation schedule is presented to other medical workers when the manager approves the change in the operation schedule. The status of each resource (resource information) may be constantly presented to the manager.

Furthermore, also in a case where the manager has received a request for an emergency operation or a notification of an emergency case, the operation schedule may be changed in response to an input made by the manager.

Note that, in a case where the operative method is changed during the operation, the process of FIG. 3 may be performed from the beginning to obtain the ideal operation flow corresponding to the changed operative method. The change in the operative method is detected by, for example, a voice instruction from the operator.

### <3. Exemplary Presentation of Operation Schedule>

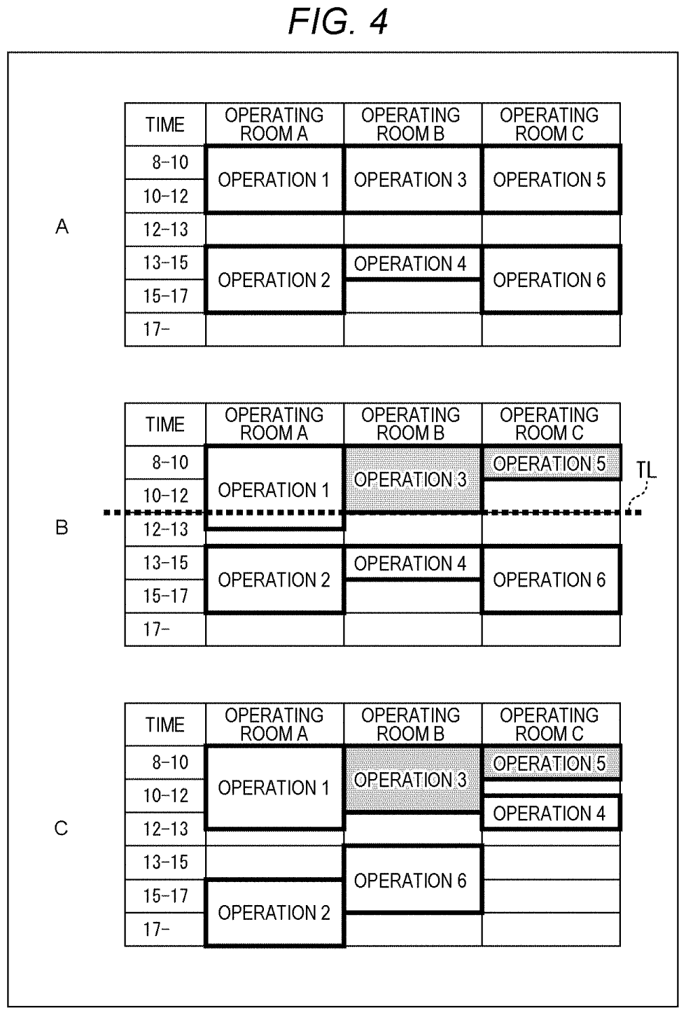

Next, exemplary presentation of an operation schedule will be described with reference to FIGS. 4 to 8.

FIG. 4 illustrates exemplary presentation of a changed operation schedule.

In FIG. 4, A represents a pre-change operation schedule retained in the database 20. In the example of FIG. 4, operation schedules for three operating rooms A, B, and C are presented for each time slot of 8:00 to 10:00, 10:00 to 12:00, 12:00 to 13:00, 13:00 to 15:00, 15:00 to 17:00, and after 17:00.

Specifically, in the operating room A, operation 1 is scheduled to be performed in the time slot of 8:00 to 12:00,and operation 2 is scheduled to be performed in the time slot of 13:00 to 17:00. In the operating room B, operation 3 is scheduled to be performed in the time slot of 8:00 to 12:00, and operation 4 is scheduled to be performed in the time slot of 13:00 to 15:00. In the operating room C, operation 5 is scheduled to be performed in the time slot of 8:00 to 12:00, and operation 6 is scheduled to be performed in the time slot of 13:00 to 17:00.

At the start time of the operation, a timeline TL indicating the current operation flow position is displayed in the operation schedule as illustrated in B of FIG. 4. In the example of B of FIG. 4, the operation flow position is 12:00.

Here, operation 3 performed in the operating room B and operation 5 performed in the operating room C have been complete at the time of 12:00, and are displayed in gray. In particular, operation 5 performed in the operating room C was complete at 10:00 earlier than the scheduled time. On the other hand, the operation flow position of operation 1 performed in the operating room A is temporally delayed.

In this case, the operation schedules in the operating rooms A, B, and C are changed as illustrated in C of FIG. 4.

Specifically, operation 2 scheduled to be performed in the operating room A from 13:00 is changed to be performed from 15:00. Operation 4 scheduled to be performed in the operating room B from 13:00 is changed to be performed in the operating room C from 11:00. Operation 6 scheduled to be performed in the operating room C from 13:00 is changed to be performed in the operating room B from 13:00.

The reason why operation 2 is started with a delay without changing the operating room is that operation 2 can be performed only in the operating room A due to equipment and other reasons. Furthermore, the reason why operation 4 is changed to be performed in the operating room C, where operation 5 has been complete at 10:00 earlier than the scheduled time, is that operation 4 may be delayed. Accordingly, operation 6 is changed to be performed in the operating room B.

In this manner, the operation schedule is changed according to the status (progress) of the operation.

Furthermore, in a case where the operation flow position is determined to be temporally delayed, the schedule management unit 32 may calculate an extendable time slot of the operation, and the calculated extendable time slot may be reflected in the operation schedule. The extendable time slot indicates a time slot in which resources including, for example, medical workers such as surgeons and nurses, medical instruments, and an operating room involved in the operation can be extended to be involved in the operation.

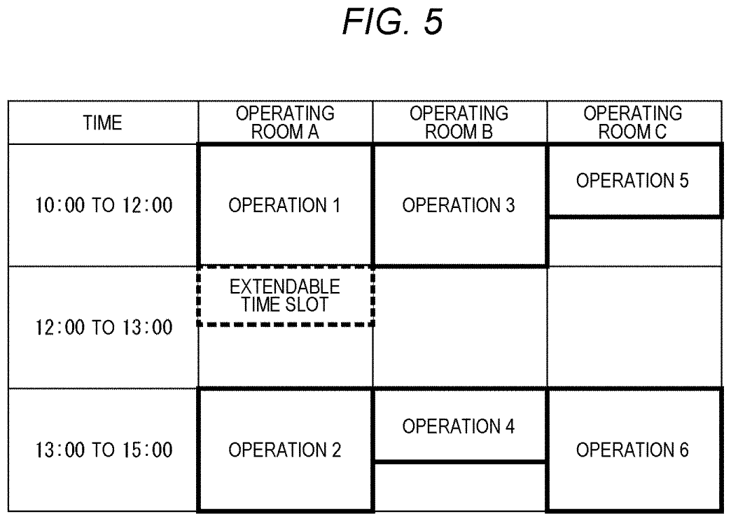

FIG. 5 illustrates the operation schedule same as that of A in FIG. 4.

In the example of FIG. 5, the operation flow position of operation 1 being performed in the operating room A is determined to be temporally delayed, whereby the extendable time slot for operation 1 (12:00 to 12:30) is presented.

Therefore, the medical workers involved in operation 2 to be performed in the operating room A subsequent to operation 1 can adjust the timing of starting preparation for the operation and the like.

Furthermore, the usage status of the medical instrument according to the type of the operation currently in progress may be reflected in the operation schedule presented to the presentation unit.

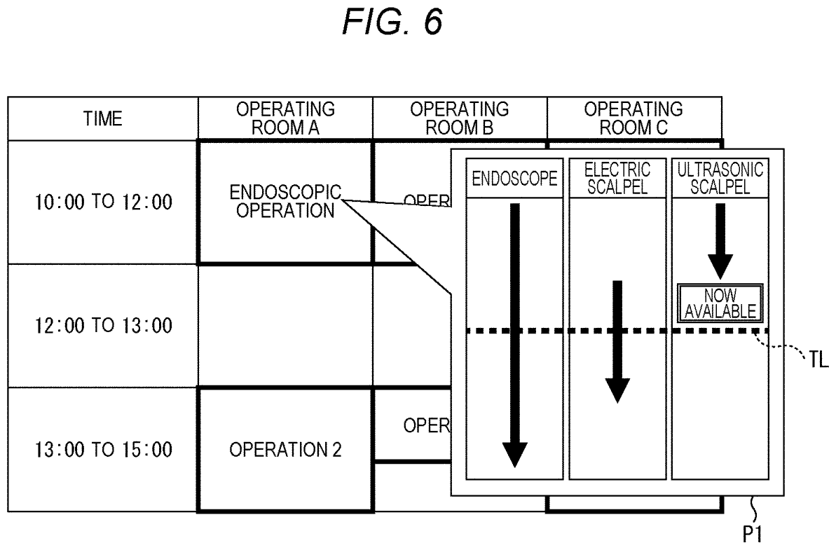

For example, in the operation schedule illustrated in FIG. 6, when a user selects the endoscopic operation being performed in the operating room A, a pop-up window P1 is displayed. The pop-up window P1 displays usage statuses of three medical instruments (endoscope, electric scalpel, and ultrasonic scalpel) used for the endoscopic operation. Furthermore, the pop-up window P1 displays the timeline TL indicating the current operation flow position.

In the example of FIG. 6, the endoscope is continuously used while the endoscopic operation is being performed, the electric scalpel is used in the middle of the endoscopic operation, and the ultrasonic scalpel is used only in the early stage of the endoscopic operation, which are indicated by respective arrows. Note that a message indicating that the ultrasonic scalpel is available is displayed as it is already vacant and will not be used at the present time point indicated by the timeline TL.

Furthermore, although illustration is omitted, for a medical instrument to be used only at the end of the operation, such as an automatic suturing device, a message indicating that it is available may be displayed at the early stage of the operation.

In this manner, with the usage status of medical instruments reflected in the operation schedule, medical workers involved in a certain operation can understand whether or not medical instruments used in another operation can be used.

Although operation schedules of operating rooms in one hospital are supposed to be presented in the above descriptions, schedules of rooms in nearby medical facilities may be presented.

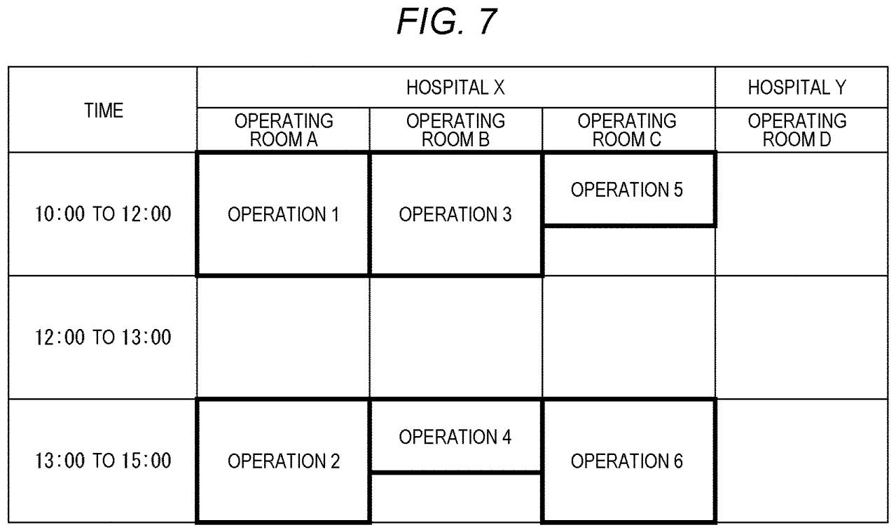

For example, in the operation schedule illustrated in FIG. 7, a schedule of an operating room D in a hospital Y is presented in addition to schedules of operating rooms A, B, and C in a hospital X. With this arrangement, any of operations 1 to 6 to be performed in the operating rooms A, B, and C in the hospital X can be changed to the operating room D in the hospital Y.

<4. Variations>

Hereinafter, variations of the hospital system 1 will be described.

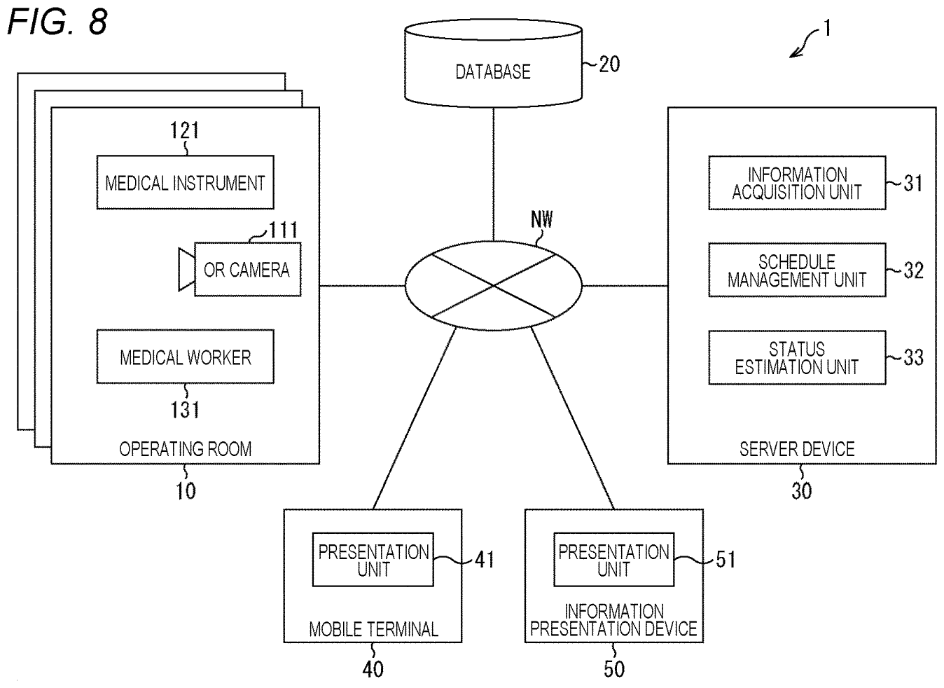

FIG. 8 illustrates a first variation of the hospital system 1.

In FIG. 8, the operating room 10 includes an operation room (OR) camera 111.

The OR camera 111 images a medical instrument 121 and a medical worker 131 in the operating room 10.

Furthermore, although illustration is omitted, the operating room 10 includes an information processing apparatus having a function similar to that of the information processing apparatus 12 in FIG. 1. By analyzing the image captured by the OR camera 111, the information processing apparatus transmits resource information such as the medical instrument 121 and its operational status in the operating room 10 and placement of the medical worker 131 to the server device 30 via the network NW.

The medical instrument 121 and the medical worker 131 may have a configuration that facilitates analysis of the image captured by the OR camera 111, which eventually facilitates acquisition of resource information. For example, the medical instrument 121 may have a label on which a two-dimensional bar code is printed, a light-emitting diode (LED) lamp, a small monitor, or the like, and the medical worker 131 may have an ID card on which a two-dimensional bar code is printed or the like.

With the configuration described above as well, it becomes possible to update the operation schedule appropriately even in a case where a significant extension occurs in the operation.

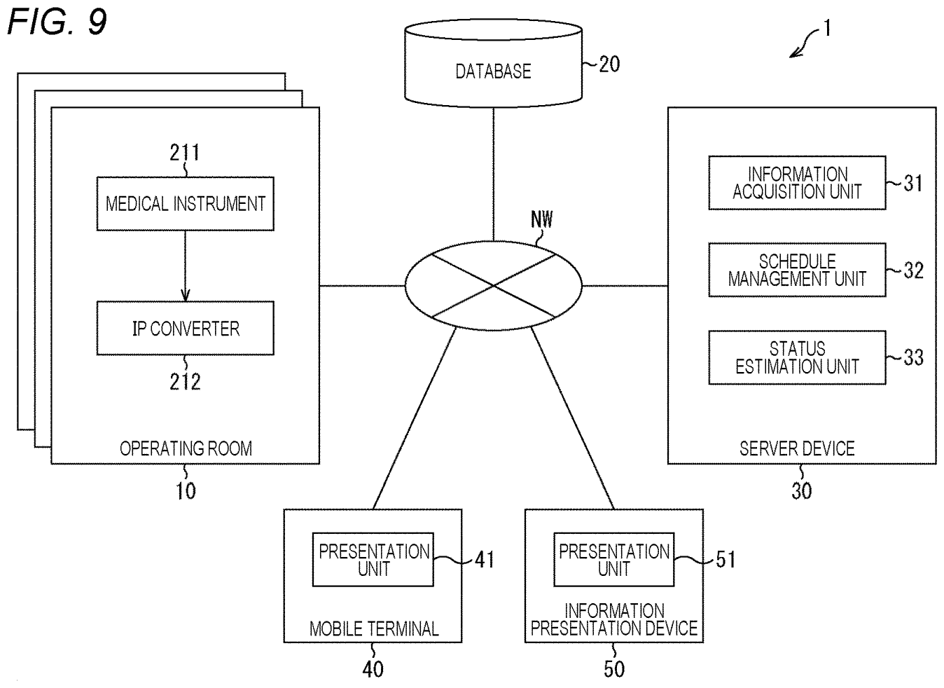

FIG. 9 illustrates a second variation of the hospital system 1.

In FIG. 8, the operating room 10 includes a medical instrument 211 and an IP converter 212.

The IP converter 212 performs IP conversion on various images from the medical instrument 211 such as an operative field camera, an endoscope, and a microscope, and transmits them to the server device 30 via the network NW.

Furthermore, although illustration is omitted, the operating room 10 includes an information processing apparatus having a function similar to that of the information processing apparatus 12 in FIG. 1. The information processing apparatus transmits resource information such as the medical instrument 121 and its operational status in the operating room and placement of the medical worker 131 to the server device 30 via the network NW.

With the configuration described above as well, it becomes possible to update the operation schedule appropriately even in a case where a significant extension occurs in the operation.

Furthermore, with the use of the IP converter, the operating room can be constructed with simple wiring, and low-delay and high-quality image transmission is enabled to support safe and reliable progress in the operation.

<5. Hardware Configuration>

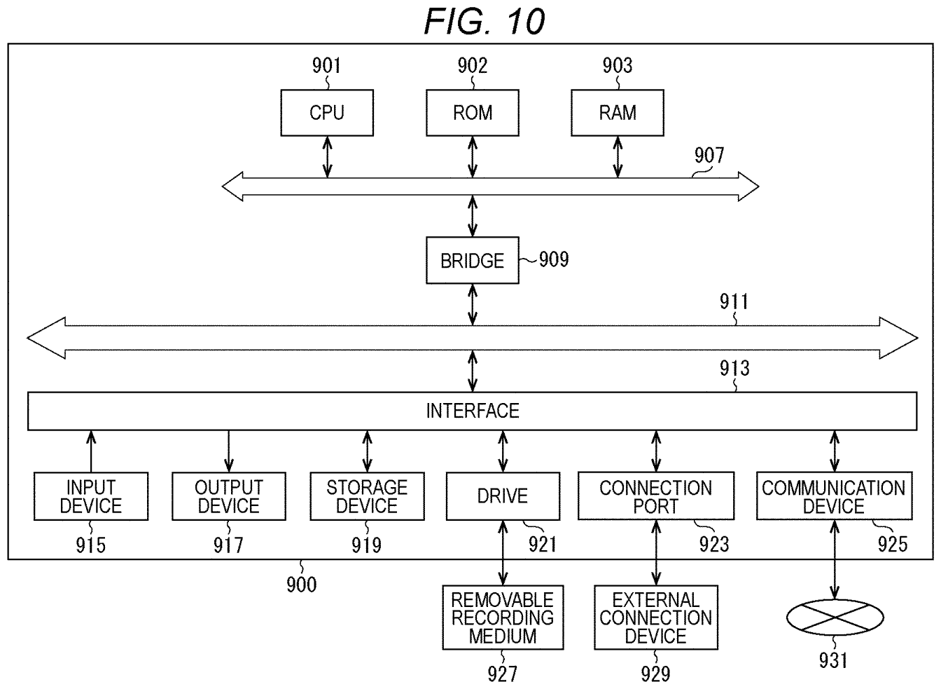

Next, an exemplary hardware configuration of the server device included in the hospital system according to the present embodiment will be described with reference to FIG. 10.

FIG. 10 is a block diagram illustrating exemplary hardware configuration of a server device 900 included in the hospital system according to the present embodiment.

As illustrated in FIG. 10, the server device 900 includes a central processing unit (CPU) 901, a read-only memory (ROM) 903, and a random access memory (RAM) 905. Moreover, the server device 900 includes a host bus 907, a bridge 909, an external bus 911, an interface 913, an input device 915, an output device 917, and a storage device 919. Note that the server device 900 may include a drive 921, a connection port 923, and a communication device 925.

The CPU 901 functions as an arithmetic processing unit and a control unit, and controls overall operation in the server device 900 or a part thereof in accordance with various programs recorded in the ROM 903, the RAM 905, the storage device 919, or a removable recording medium 927.

The ROM 903 stores programs, operation parameters, and the like to be used by the CPU 901. The RAM 905 temporarily stores programs to be used by the CPU 901, parameters that appropriately change in the execution of the programs, and the like. These are mutually connected by the host bus 907 including an internal bus such as a CPU bus. Note that each configuration of the server device 30 described with reference to FIG. 1 is implemented by, for example, the CPU 901.

The host bus 907 is connected to the external bus 911, such as a peripheral component interconnect/interface (PCI) bus, via the bridge 909. The input device 915, the output device 917, the storage device 919, the drive 921, the connection port 923, and the communication device 925 are connected to the external bus 911 via the interface 913.

The input device 915 is an operation means to be operated by the user, which is, for example, a mouse, a keyboard, a touch panel, a button, a switch, a lever, a pedal, and the like. Furthermore, the input device 915 may be, for example, a remote control means (what is called a remote controller) using infrared rays or other radio waves, or may be an external connection device 929 such as a mobile phone and a personal digital assistant (PDA) supporting operation of the server device 900.

The input device 915 includes, for example, an input control circuit or the like that generates input signals on the basis of information input by the user using the operation means mentioned above and outputs the signals to the CPU 901.

The user operates the input device 915 to input various data or to provide an instruction for processing operation to the server device 900.

The output device 917 includes a device capable of visually or aurally notifying the user of the obtained information. Specifically, the output device 917 is configured as a display device such as a cathode-ray tube (CRT) display device, a liquid crystal display device, a plasma display device, an electroluminescent (EL) display device, and a lamp, an audio output device such as a speaker and a headphone, a printer device, or the like.

The output device 917 outputs, for example, a result obtained by various kinds of processing performed by the server device 900. Specifically, the display device displays the result obtained by various kinds of processing performed by the server device 900 as text or an image. Meanwhile, the audio output device converts audio signals including reproduced voice data, acoustic data, and the like into analog signals and outputs the analog signals.

The storage device 919 is a device for storing data, which is an example of a storage unit of the server device 900. The storage device 919 includes, for example, a magnetic storage unit device such as a hard disk drive (HDD), a semiconductor storage device, an optical storage device, a magneto-optical storage device, or the like. The storage device 919 stores programs to be executed by the CPU 901, various data, and the like.

The drive 921 is a reader/writer for a recording medium, which is incorporated in or externally attached to the server device 900. The drive 921 reads information recorded in the placed removable recording medium 927 such as a magnetic disk, an optical disk, a magneto optical disk, a semiconductor memory, or the like and outputs the information to the RAM 905. Furthermore, the drive 921 is also capable of writing a record to the placed removable recording medium 927 such as a magnetic disk, an optical disk, a magneto optical disk, a semiconductor memory, or the like.

The removable recording medium 927 is, for example, digital versatile disc (DVD) media, HD-DVD media, Blu-ray (registered trademark) media, or the like. Furthermore, the removable recording medium 927 may be a CompactFlash (CF) (registered trademark), a flash memory, a secure digital (SD) memory card, or the like. Moreover, the removable recording medium 927 may be, for example, an integrated circuit (IC) card equipped with a non-contact IC chip, an electronic device, or the like.

The connection port 923 is a port for directly connecting the external connection device 929 to the server device 900. Examples of the connection port 923 include a universal serial bus (USB) port, an IEEE 1394 port, and a small computer system interface (SCSI) port. Other examples of the connection port 923 include an RS-232C port, an optical audio terminal, and a high-definition multimedia interface (HDMI) (registered trademark) port. With the external connection device 929 connected to the connection port 923, the server device 900 obtains various data directly from the external connection device 929, and provides various data to the external connection device 929.

The communication device 925 is, for example, a communication interface including a communication device or the like for connecting to a communication network 931. The communication device 925 is, for example, a communication card for wired or wireless local area network (LAN), Bluetooth (registered trademark), wireless USB (WUSB), or the like. Furthermore, the communication device 925 may be a router for optical communication, a router for asymmetric digital subscriber line (ADSL), a modem for various kinds of communication, or the like.

For example, the communication device 925 is capable of transmitting and receiving signals using, for example, a predetermined protocol such as TCP/IP with the Internet and other communication equipment. Furthermore, the communication network 931 to be connected to the communication device 925 may include a network connected by wire or wirelessly. The communication network 931 may be, for example, the Internet or a home LAN, or may be a communication network to perform infrared communication, radio wave communication, or satellite communication.

Each component of the server device 900 described above may be configured using a general-purpose member, or may be configured by hardware specialized for the function of each component. Therefore, it is possible to appropriately change the hardware configuration to be used depending on the technical level of the time of carrying out the present embodiment.

Moreover, it is possible to create a computer program for implementing each function of the server device 900 included in the hospital system according to the present embodiment to implement it on a personal computer or the like. Furthermore, it is also possible to provide a computer-readable recording medium storing such a computer program. The recording medium is, for example, a magnetic disk, an optical disk, a magneto optical disk, a flash memory, or the like. Furthermore, the computer program may be distributed via a network, for example, without using a recording medium.

Note that the information processing apparatus 12 included in the hospital system according to the present embodiment can also be configured in a similar manner to the server device 900 of FIG. 10.

<6. Summary>

In the hospital system according to the present embodiment, the following items can be achieved.

(1) Optimal review of operation schedule in the case of extending operation

(2) Notification of estimated operation end time to anesthesiologists and pathologists

(3) Efficient operation of resources (room, equipment, and manpower)

(4) Efficient transfer of equipment and people

(5) Optimal review of emergency operation schedule and reduction of impact on other operations
Physical change in a patient
Medical instrument failure
Change in physical conditions of a medical worker
Emergency case

(6) Cooperation with nearby medical facilities

According to the hospital system according to the present embodiment, it becomes possible to efficiently manage and use medical instruments among a plurality of operating rooms. This not only improves operations, but also eliminates factors such as duplicate reservations and misplacement of medical instruments, whereby safer medical care with improved service of medical workers and excellent medical economic efficiency can be provided.

Note that the embodiment of the present disclosure is not limited to the embodiments described above, and various modifications can be made without departing from the gist of the present disclosure.

For example, the present disclosure may employ a configuration of cloud computing in which one function is shared and jointly processed by a plurality of devices via a network.

Furthermore, each step described in the flowchart described above can be executed by one device or shared by a plurality of devices.

Moreover, in a case where a plurality of processes is included in one step, the plurality of processes included in the one step can be executed by one device or shared by a plurality of devices.

Furthermore, the present disclosure can employ the following configurations.

(1)

A hospital system including:

a schedule management unit that manages an operation schedule;

a presentation unit that presents the operation schedule; and

a status estimation unit that estimates, on the basis of a type of an operation currently in progress and resource information from a resource related to the operation, a status of the operation, in which

the schedule management unit reflects the estimated status of the operation in the operation schedule, and

the presentation unit presents the operation schedule in which the status of the operation is reflected.

(2)

The hospital system according to (1), in which

the resource information includes equipment information from a medical instrument used for the operation.

(3)

The hospital system according to (2), in which the equipment information includes an operative field image captured by an operative field camera.

(4)

The hospital system according to (2), in which the equipment information includes on/off information of the medical instrument.

(5)

The hospital system according to (1), in which the resource information includes input information input by a medical worker.

(6)

The hospital system according to (5), in which the input information includes a voice instruction from the medical worker.

(7)

The hospital system according to any one of (1) to (6), in which

the status estimation unit estimates, as the status of the operation, an operation flow position that is a current temporal position in an operation flow, and

the presentation unit presents the operation schedule in which the operation flow position is reflected.

(8)

The hospital system according to (7), in which the schedule management unit changes the operation schedule according to the estimated operation flow position, and

the presentation unit presents the changed operation schedule.

(9)

The hospital system according to (7), in which

the schedule management unit calculates an extendable time slot of the operation in a case where the operation flow position is temporally delayed compared to an ideal operation flow position corresponding to the type of the operation, and

the presentation unit presents the operation schedule in which the extendable time slot is reflected.

(10)

The hospital system according to (7), in which

the schedule management unit generates an alternative schedule of a next operation to be performed temporally later than the operation currently in progress in a case where the operation flow position is temporally delayed compared to an ideal operation flow position corresponding to the type of the operation, and

the presentation unit presents the operation schedule in which the alternative schedule is reflected.

(11)

The hospital system according to claim (10), in which

the schedule management unit generates the alternative schedule on the basis of a priority level of the next operation.

(12)

The hospital system according to (11), in which

the schedule management unit determines the priority level according to a status of the resource related to the next operation.

(13)

The hospital system according to any one of (1) to (12), in which

the presentation unit presents the operation schedule in which a usage status of a medical instrument according to the type of the operation is reflected.

(14)

The hospital system according to (13), in which

the presentation unit presents availability of the medical instrument in another operation based on the usage status of the medical instrument.

(15)

The hospital system according to any one of (1) to (14), in which

the presentation unit presents the resource required for the operation in a case where the type of the operation is input.

(16)

The hospital system according to (15), in which

the presentation unit presents an existing position of the resource required for the operation on the basis of positional information of the resource.

(17)

A server device including:

a schedule management unit that manages an operation schedule; and

a status estimation unit that estimates, on the basis of a type of an operation currently in progress and resource information from a resource related to the operation, a status of the operation, in which

the schedule management unit reflects the estimated status of the operation in the operation schedule.

(18)

A method of managing a schedule causing a server device that manages an operation schedule to perform:

estimating, on the basis of a type of an operation currently in progress and resource information from a resource related to the operation, a status of the operation; and

reflecting the estimated status of the operation in the operation schedule.

### REFERENCE SIGNS LIST

1 Hospital system

10 Operating room

11 Medical instrument

12 Information processing apparatus

12c Presentation unit

20 Database

30 Server device

31 Information acquisition unit

32 Schedule management unit

33 Status estimation unit

40 Mobile terminal

41 Presentation unit

50 Information presentation device

51 Presentation unit

1. A hospital system comprising:
a schedule management unit that manages an operation schedule;
a presentation unit that presents the operation schedule; and
a status estimation unit that estimates, on a basis of a type of an operation currently in progress and resource information from a resource related to the operation, a status of the operation, wherein
the schedule management unit reflects the estimated status of the operation in the operation schedule, and
the presentation unit presents the operation schedule in which the status of the operation is reflected.

2. The hospital system according to claim 1, wherein
the resource information includes equipment information from a medical instrument used for the operation.

3. The hospital system according to claim 2, wherein
the equipment information includes an operative field image captured by an operative field camera.

4. The hospital system according to claim 2, wherein
the equipment information includes on/off information of the medical instrument.

5. The hospital system according to claim 1, wherein
the resource information includes input information input by a medical worker.

6. The hospital system according to claim 5, wherein
the input information includes a voice instruction from the medical worker.

7. The hospital system according to claim 1, wherein
the status estimation unit estimates, as the status of the operation, an operation flow position that is a current temporal position in an operation flow, and
the presentation unit presents the operation schedule in which the operation flow position is reflected.

8. The hospital system according to claim 7, wherein
the schedule management unit changes the operation schedule according to the estimated operation flow position, and
the presentation unit presents the changed operation schedule.

9. The hospital system according to claim 7, wherein
the schedule management unit calculates an extendable time slot of the operation in a case where the operation flow position is temporally delayed compared to an ideal operation flow position corresponding to the type of the operation, and
the presentation unit presents the operation schedule in which the extendable time slot is reflected.

10. The hospital system according to claim 7, wherein
the schedule management unit generates an alternative schedule of a next operation to be performed temporally later than the operation currently in progress in a case where the operation flow position is temporally delayed compared to an ideal operation flow position corresponding to the type of the operation, and
the presentation unit presents the operation schedule in which the alternative schedule is reflected.

11. The hospital system according to claim 10, wherein
the schedule management unit generates the alternative schedule on a basis of a priority level of the next operation.

12. The hospital system according to claim 11, wherein
the schedule management unit determines the priority level according to a status of the resource related to the next operation.

13. The hospital system according to claim 1, wherein
the presentation unit presents the operation schedule in which a usage status of a medical instrument according to the type of the operation is reflected.

14. The hospital system according to claim 13, wherein
the presentation unit presents availability of the medical instrument in another operation based on the usage status of the medical instrument.

15. The hospital system according to claim 1, wherein
the presentation unit presents the resource required for the operation in a case where the type of the operation is input.

16. The hospital system according to claim 15, wherein
the presentation unit presents an existing position of the resource required for the operation on a basis of positional information of the resource.

17. A server device comprising:
a schedule management unit that manages an operation schedule; and
a status estimation unit that estimates, on a basis of a type of an operation currently in progress and resource information from a resource related to the operation, a status of the operation, wherein
the schedule management unit reflects the estimated status of the operation in the operation schedule.

18. A method of managing a schedule causing a server device that manages an operation schedule to perform:
estimating, on a basis of a type of an operation currently in progress and resource information from a resource related to the operation, a status of the operation; and
reflecting the estimated status of the operation in the operation schedule.
